(19) United States
(12) Patent Application Publication
(10) Pub. No.: US 2026/0054099 A1
(43) Pub. Date: Feb. 26, 2026

(54) DEVICE COMPRISING AT LEAST ONE ULTRASONIC VIBRATOR, AND METHOD FOR CONTROLLING SAME

(71) Applicant: LG ELECTRONICS INC., Seoul (KR)

(72) Inventors: Sangwon KIM, Seoul (KR); Yongju YANG, Seoul (KR); Gyuseog HONG, Seoul (KR); Munseong KANG, Seoul (KR); Seungyoon SHIN, Seoul (KR); Dongwon KIM, Seoul (KR)

(73) Assignee: LG ELECTRONICS INC., Seoul (KR)

(21) Appl. No.: 19/103,973

(22) PCT Filed: Aug. 22, 2023
(86) PCT No.: PCT/KR2023/012379

§ 371 Date: Feb. 14, 2025

(30) Foreign Application Priority Data
Aug. 22, 2022 (KR) 10-2022-0104494

(51) Int. Cl. A61N 7/00

(52) U.S. Cl. CPC A61N 7/00; A61N 2007/0034

(57) ABSTRACT

A method for controlling a device comprising at least one ultrasonic vibrator according to one embodiment of the present disclosure comprises the steps of: recognizing whether there is an abnormality in the at least one ultrasonic vibrator; if no abnormality is found as a result of the recognition, determining whether target energy compensation of the at least one ultrasonic vibrator is necessary; and if compensation is found to be necessary as a result of the determination, increasing the voltage input to a pulser, increasing PWM duty ratio, or increasing emission time.

### TECHNICAL FIELD

Ultrasound is a vibration sound wave in a band that is not able to be heard by human ears, and refers to a vibration sound wave generated when electrical energy generated by a potential difference between both ends of an ultrasonic vibrator is converted into mechanical energy. For example, 1 Mhz, which is a basic unit of therapeutic ultrasound used in this patent, means vibrating a million times per second, and 3 Mhz means vibrating 3 million times per second.

The present disclosure is applicable to all types of devices including at least one ultrasonic vibrator. The present disclosure is not limited to a cosmetology device, a medical device, and the like.

### BACKGROUND

Ultrasound originated in 1877 when the Curie brothers discovered a piezoelectric effect, where applying a potential difference signal to both ends of a crystal or barium material causes a mechanical change, and such effect was applied to a SONAR.

Application of the ultrasound to a human body began in a medical field, utilizing vibrational sound wave energy generated by a reverse piezoelectric effect. Today, the ultrasound is widely applied across various industries.

A propagation speed of the ultrasound varies depending on a hardness and a density of a medium, causing a difference in a reflected wave of the sound wave. Lower frequencies penetrate deeper and farther, while higher frequencies experience greater attenuation and operate more actively at a surface level. Typically, ultrasound devices are classified into a diagnostic ultrasound device used for diagnosis and identification of an internal organ, a blood vessel, and a lesion, and a therapeutic ultrasound device aimed at physical improvement and pain relief.

However, according to prior art, in case of a device including an ultrasonic vibrator, because a manufacturing resonant frequency is applied regardless of an individual resonant frequency of each vibrator, a resonant frequency error occurs in each vibrator.

In addition, when a voltage input to the ultrasonic vibrator is different from a target output, there is a disadvantage in that there is no feedback circuit capable of compensating therefor.

In addition, when a plurality of ultrasonic vibrators are used, when a small defect occurs in one ultrasonic vibrator, a voltage and a current do not flow properly in a defective vibrator, so that the target output is not achieved and thus a treatment effect is halved. In particular, because the ultrasonic vibrator is made of a fragile material, a solution for determining whether the vibrator is broken is required.

### SUMMARY

### Technical Problem

According to one of embodiments of the present disclosure, it is to provide a device including a circuit and a function for receiving current feedback of an output applied to an ultrasonic vibrator.

According to one of embodiments of the present disclosure, it is to provide a device that may perform energy compensation by adaptively adjusting a parameter. For example, it is to provide a function of correcting an ultrasonic vibrator output voltage by applying an adaptive envelope circuit or the like to measure a potential difference between both ends of an ultrasonic vibrator.

### Technical Solutions

A method for controlling a device including at least one ultrasonic vibrator according to one of embodiments of the present disclosure includes recognizing whether the at least one ultrasonic vibrator is abnormal, determining whether target energy compensation of the at least one ultrasonic vibrator is required when no abnormality is found as a result of the recognition, and increasing a voltage input to a pulser, increasing a pulse width modulation (PWM) duty ratio, or increasing an irradiation time when the compensation is required as a result of the determination.

A device according to one of embodiments of the present disclosure includes at least one ultrasonic vibrator, a pulser that creates an ultrasonic waveform, and a controller that recognizes whether the at least one ultrasonic vibrator is abnormal, determines whether target energy compensation of the at least one ultrasonic vibrator is required when no abnormality is found as a result of the recognition, and increases a voltage input to the pulser, increases a PWM duty ratio, or increases an irradiation time when the compensation is required as a result of the determination.

Further, a cosmetic care device according to one of embodiments of the present disclosure includes a head including at least one skin temperature sensor and at least one ultrasonic vibrator, and a main body including a pulser that creates an ultrasonic waveform, and a controller that controls the at least one ultrasonic vibrator included in the head. Particularly, when a specific ultrasonic vibrator among the at least one ultrasonic vibrator is predicted to reach a temperature equal to or higher than a predetermined temperature, the controller lowers a voltage input to the pulser and increases an irradiation time of the specific ultrasonic vibrator such that the specific ultrasonic vibrator is operated at a temperature lower than the predetermined temperature.

### Advantageous Effects

According to one of the embodiments of the present disclosure, the surface temperature of the ultrasonic vibrator over time may be automatically estimated in consideration of the difference between the initial input high voltage applied to the ultrasonic pulse generator and the high voltage applied to the actual vibrator. To implement the above, for example, the ultrasonic vibrator temperature table is stored in the memory (e.g., the ROM) of the MCU, which is the controller, or the external flash memory in the form of the LUT.

According to one of the embodiments of the present disclosure, the surface temperature of the ultrasonic vibrator may be estimated using the algorithm for comparing the current consumption based on the input voltage difference with the vibrator temperature table. Furthermore, the algorithm for increasing the irradiation time until reaching the target energy amount after the voltage compensation up to about 41 degrees when the surface temperature of the arbitrary ultrasonic vibrator among the plurality of ultrasonic vibrators is predicted to be equal to or higher than 43 degrees may be applied.

According to one of the embodiments of the present disclosure, whether the ultrasonic vibrator is abnormal may be identified by comparing the input voltage and the output voltage of the ultrasonic vibrator with each other. For example, when the input/output voltage difference is equal to or greater than 10V, it is considered that there is the abnormality in the vibrator.

According to one of the embodiments of the present disclosure, provided is the device for identifying whether the patch is connected and performing the operation-related control using the current Feedback.

In addition, according to one of the embodiments of the present disclosure, provided is the device having the output automatic correction function using the output power correction algorithm.

In addition to the technical effects described above, it is obvious that the effect that the person skilled in the art may infer through the present document should also be considered.

### BRIEF DESCRIPTION OF THE DRAWINGS

FIG. 1 illustrates an entire system according to one of embodiments of the present disclosure.

FIG. 2 is a block diagram illustrating only main blocks of a device according to one of embodiments of the present disclosure.

FIG. 3 is a diagram for illustrating a process of comparing input/output voltages applied to an ultrasonic vibrator with each other according to one of embodiments of the present disclosure.

FIG. 4 illustrates an embodiment of a device using a vibrator temperature estimation LUT.

FIG. 5 is a flowchart illustrating a method for controlling a device according to an embodiment of the present disclosure.

Further, FIG. 6 illustrates an embodiment of a device that uses frequency shifting for a resonant frequency.

### BEST MODE

Hereinafter, embodiments disclosed herein will be described in detail with reference to the accompanying drawings. The same or similar components will be given the same reference numerals regardless of the drawings, and redundant descriptions thereof will be omitted. The suffixes “module” and “unit” for the components used in the following description are given or used interchangeably in consideration of only the ease of writing the present document, and do not have a distinct meaning or role.

In addition, in describing the embodiments disclosed herein, when it is determined that a detailed description of the related known technology may obscure the gist of the embodiments disclosed herein, the detailed description thereof will be omitted. In addition, the accompanying drawings are only for easily understanding of the embodiments disclosed herein, and the technical spirit disclosed herein is not limited by the accompanying drawings, and should be understood to include all changes, equivalents, and substitutes included in the spirit and technical scope of the present disclosure.

Terms including an ordinal number, such as first, second, and the like, may be used to describe various components, but the components are not limited by the terms. The terms are used only for the purpose of distinguishing one component from another component. When it is mentioned that a component is “connected” or “linked” to another component, it should be understood that the components may be directly connected or linked to each other, but a third component may be present therebetween.

On the other hand, when it is mentioned that a component is “directly connected” or “directly linked” to another component, it should be understood that a third component is not present therebetween.

A singular expression includes a plural expression unless the context clearly dictates otherwise.

As used herein, it should be understood that terms such as “include” or “have” are intended to specify the presence of features, numbers, steps, operations, components, parts, or combinations thereof described herein, and do not preclude the presence or addition of one or more other features, numbers, steps, operations, components, parts, or combinations thereof.

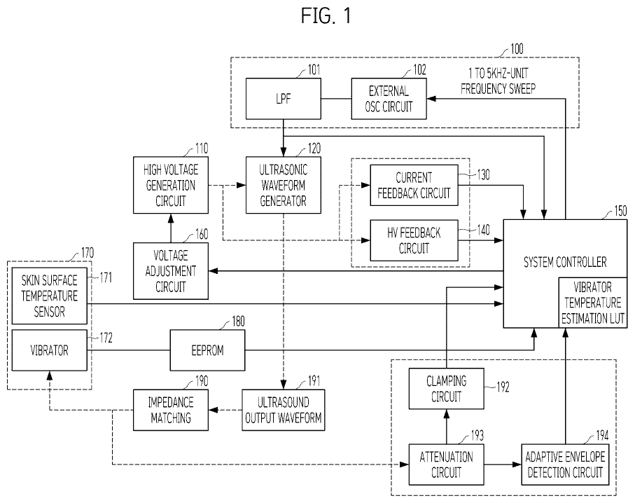

FIG. 1 illustrates an entire system according to one of embodiments of the present disclosure. The system according to the present disclosure may be used for body care, medical treatment, and the like, but may be applied to any type of device using an ultrasonic vibrator.

FIG. 1 illustrates an overall configuration of the device, but the present disclosure is not necessarily limited thereto, and should be determined based on matters described in the claims.

The entire system includes a frequency modulator 100, a high voltage generation circuit 110, an ultrasonic waveform generator 120, a current feedback circuit 130, a high voltage feedback circuit 140, a system controller 150, a voltage adjustment circuit 160, a head 170, an EEPROM 180, an impedance matching circuit 190, an ultrasound output waveform 191, a clamping circuit 192, an attenuation circuit 193, an adaptive envelope detection circuit 194, and the like.

The frequency modulator 100 described above may be composed of, for example, an LPF 101 for signal clearance, an external OSC circuit 102, and the like.

The head 170 described above is composed of, for example, a skin surface temperature sensor 171, a vibrator 172, and the like. Herein, the vibrator 172 corresponds to, for example, one or more ultrasonic vibrators.

Replacing the above-described EEPROM 180 with another memory also belongs to another scope of the present disclosure.

The current feedback circuit 130 and the high voltage feedback circuit 140 sense a current and a voltage input to the ultrasonic waveform generator 120 and transmit the sensed current and voltage to the system controller 150.

The voltage adjustment circuit 160 may perform a function of adjusting the voltage using a variable resistor.

The EEPROM 180 stores information on the ultrasonic vibrator.

The impedance matching circuit 190 is based on the most frequently used LC tuning, which may be changed to CLC tuning. Further, the ultrasound output waveform 191 may be, for example, a rectangular wave or a sine wave (30-80V).

The clamping circuit 192 is a circuit that clips an input higher than 3.3V into an 3.3V waveform.

The attenuation circuit 193 is a circuit that reduces an output waveform voltage to 1/40 of an original value.

The adaptive envelope detection circuit senses an output frequency and an output voltage applied to the ultrasonic vibrator and transmits the sensed output frequency and output voltage to the system controller 150.

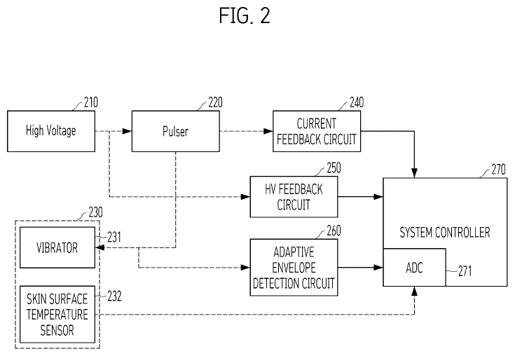

FIG. 2 is a block diagram illustrating only main blocks of a device according to one of embodiments of the present disclosure. For example, some modules in the entire system shown in FIG. 1 are deleted and then the system is simplified in FIG. 2.

However, the device according to the present disclosure does not necessarily use all of the blocks illustrated in FIG. 2, and addition, deletion, or replacement of some blocks also belongs to another scope of the present disclosure.

As illustrated in FIG. 2, the device according to an embodiment of the present disclosure includes a high voltage generation circuit 210, a pulser 220, a vibrator 231, a skin temperature sensor 232, a current feedback circuit 240, a high voltage feedback circuit 250, an adaptive envelope detection circuit 260, a system controller 270, and the like.

The vibrator 231 and the skin temperature sensor 232 may be included in a head 230, and the remaining components may be included in a main body, but the present disclosure may not be necessarily limited thereto.

Furthermore, the system controller 270 may additionally include an ADC 271. Here, the system controller 270 may be various controllers such as an MCU. The ADC 271 means an analog to digital converter.

An embodiment of the present disclosure will be described in more detail as follows.

First, the system controller 270 is designed to compare a high voltage input to the pulser 220 that creates an ultrasonic waveform with a high voltage applied to and output from the actual vibrator 231.

When a difference between the input applied to the pulser 220 and the input voltage applied to the actual vibrator 231 is equal to or smaller than about 0.3V, the device is designed to determine that a patch is not connected to the system, and not to operate a treatment process.

Furthermore, the system controller 270 identifies whether a currently output energy amount is equal to a target energy amount by applying the actually input voltage and a feedback current to an algorithm.

For example, when the energy amount is smaller than 100 mW/cm2, the input voltage applied to the vibrator 231 is compensated for adaptively via comparison with a voltage-temperature table.

In addition, when the difference between the input high voltage input to the pulser 220 and the voltage applied to and output from the vibrator 231 is equal to or greater than 20%, the system controller 270 determines that the vibrator 231 has failed.

Because the voltage applied to each vibrator is different, unlike an existing treatment device according to the prior art, there is a technical effect of delivering the accurate target energy amount when applying the above-described embodiment of the present disclosure.

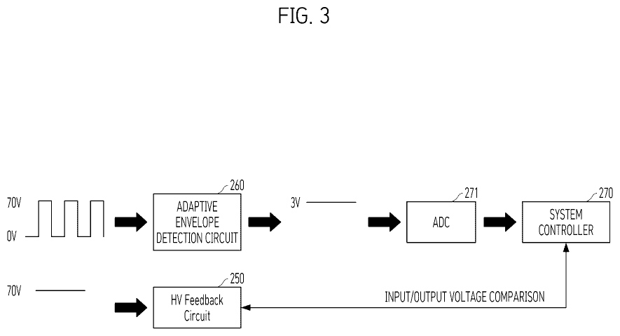

FIG. 3 is a diagram for illustrating a process of comparing input/output voltages applied to an ultrasonic vibrator with each other according to one of embodiments of the present disclosure.

First, as shown in a lower end of FIG. 3, the high voltage input to the pulser is sensed by the high voltage feedback circuit 250 and transmitted to the system controller 270.

Further, as shown in an upper end of FIG. 3, the ultrasonic wave generated via the pulser is applied to the adaptive envelope detection circuit 260 and transmitted to the system controller 270 via the ADC 271. This is a sensing result of the output voltage applied to the ultrasonic vibrator.

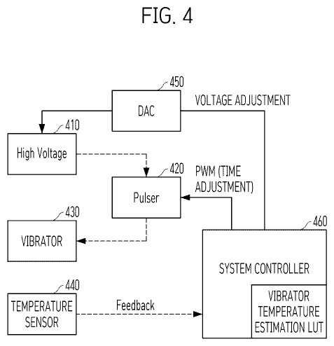

Finally, the system controller 270 may compare the initial input voltage with the output voltage applied to the ultrasonic vibrator to recognize whether there is a problem in the ultrasonic vibrator. In this regard, an LUT or the like may be used, which will be described in more detail in FIG. 4.

FIG. 4 illustrates an embodiment of a device using a vibrator temperature estimation LUT.

As illustrated in FIG. 4, the device according to an embodiment of the present disclosure includes a high voltage output unit 410, a pulser 420, a vibrator 430, a temperature sensor 440, a digital to analog converter (DAC) 450, a system controller 460, and the like.

For example, it is assumed that the device according to an embodiment of the present disclosure is used for a treatment and a specific energy amount (e.g., 100W/cm2) based on a treatment site is set.

While applying a pulse to the vibrator 430, an output and a current of the corresponding pulse are simultaneously monitored in real time via a feedback unit. When a difference from the target energy amount occurs, the system controller 460 actively adjusts a parameter.

The system controller 460 compares an input voltage applied to the pulser generation unit 420 with an output voltage applied to both ends of the vibrator 430, and estimates how much a temperature will increase using a difference therebetween. When the device according to an embodiment of the present disclosure is used for a medical purpose, it is important to design the device such that a skin surface temperature does not rise to 43 degrees or higher.

Furthermore, when it is predicted that the temperature will be equal to or higher than 43 degrees because a compensation for one ultrasonic vibrator among the plurality of ultrasonic vibrators is great, it may be designed to perform the voltage compensation only until reaching 39 degrees and increase an irradiation time until the target energy amount is reached. A skin temperature criterion may be made using an ultrasonic vibrator of the highest temperature.

According to an embodiment of the present disclosure, the temperature sensor 440 is used and the temperature rise of the ultrasonic vibrator resulted from the high voltage adjustment is predicted using the LUT. When the predicted temperature exceeds 43 degrees even for one ultrasonic vibrator, operation of the ultrasonic vibrator is temporarily stopped until the temperature is lowered, the voltage compensation is performed such that a maximum temperature does not exceed 41 degrees, and the target energy amount is reached by increasing the irradiation time.

In case of another ultrasonic vibrator, only the voltage compensation is performed based on a temperature estimation algorithm as it was.

However, to implement the above, the temperature sensor 440 measures an initial skin contact temperature, and the system controller 460 predicts a later skin surface temperature using the vibrator temperature estimation LUT. Thereafter, the temperature is then verified by the temperature sensor 440 whether it aligns with the skin surface temperature predicted based on the LUT.

For example, when the pulser input/output voltage difference is 1V and the initial temperature is 34 degrees, it is available to predict that the temperature will rise to 35.2 degrees after 10 minutes with reference to the LUT below.

Furthermore, when the pulser input/output voltage difference is 2V and the initial temperature is 34 degrees, it is available to predict that the temperature will rise to 36.4 degrees after 10 minutes with reference to the LUT illustrated in Table 1 below.

TABLE 1

| | Voltage difference | | | | | | | | | |
|---|---|---|---|---|---|---|---|---|---|---|
| | 1 | | | | | 2 | | | | |
| | Initial temperature | | | | | | | | | |
| Time | 32 | 33 | 34 | 35 | 36 | 32 | 33 | 34 | 35 | 36 |
| 1 | 32.4 | 33.3 | 34.3 | 35.4 | 36.5 | 32.5 | 33.6 | 34.6 | 35.7 | 36.5 |
| 2 | 32.5 | 33.4 | 34.4 | 35.5 | 36.6 | 32.8 | 33.8 | 34.8 | 35.9 | 36.7 |
| 3 | 32.7 | 33.5 | 34.5 | 35.6 | 36.7 | 33 | 34 | 35 | 36.2 | 36.9 |
| 4 | 32.8 | 33.6 | 34.6 | 35.7 | 36.8 | 33.2 | 34.2 | 35.2 | 36.4 | 37.2 |
| 5 | 32.9 | 33.7 | 34.7 | 35.8 | 36.9 | 33.4 | 34.4 | 35.4 | 36.6 | 37.4 |
| 6 | 33 | 33.8 | 34.8 | 35.9 | 37 | 33.6 | 34.6 | 35.6 | 36.8 | 37.6 |
| 7 | 33.2 | 33.9 | 34.9 | 36 | 37.1 | 33.8 | 34.8 | 35.8 | 36.9 | 37.8 |
| 8 | 33.4 | 34 | 35 | 36.1 | 37.2 | 34 | 35 | 36 | 37 | 38 |
| 9 | 33.7 | 34.1 | 35.1 | 36.2 | 37.3 | 34.2 | 35.2 | 36.2 | 37.2 | 38.3 |
| 10 | 33.8 | 34.2 | 35.2 | 36.3 | 37.4 | 34.4 | 35.4 | 36.4 | 37.4 | 38.4 |
| 11 | 34 | 34.4 | 35.3 | 36.4 | 37.5 | 34.6 | 35.6 | 36.5 | 37.6 | 38.6 |
| 12 | 34.2 | 34.4 | 35.4 | 36.5 | 37.5 | 34.8 | 35.8 | 36.7 | 37.8 | 38.8 |
| 13 | 34.2 | 34.4 | 35.4 | 36.5 | 37.5 | 34.8 | 35.8 | 36.7 | 37.8 | 38.8 |
| 14 | 34.2 | 34.4 | 35.4 | 36.5 | 37.5 | 34.8 | 35.8 | 36.7 | 37.8 | 38.8 |
| 15 | 34.2 | 34.4 | 35.4 | 36.5 | 37.5 | 34.8 | 35.8 | 36.7 | 37.8 | 38.8 |
| 16 | 34.2 | 34.4 | 35.4 | 36.5 | 37.5 | 34.8 | 35.8 | 36.7 | 37.8 | 38.8 |
| 17 | 34.2 | 34.4 | 35.4 | 36.5 | 37.5 | 34.8 | 35.8 | 36.7 | 37.8 | 38.8 |
| 18 | 34.2 | 34.4 | 35.4 | 36.5 | 37.5 | 34.8 | 35.8 | 36.7 | 37.8 | 38.8 |
| 19 | 34.2 | 34.4 | 35.4 | 36.5 | 37.5 | 34.8 | 35.8 | 36.7 | 37.8 | 38.8 |
| 20 | 34.2 | 34.4 | 35.4 | 36.5 | 37.5 | 34.8 | 35.8 | 36.7 | 37.8 | 38.8 |

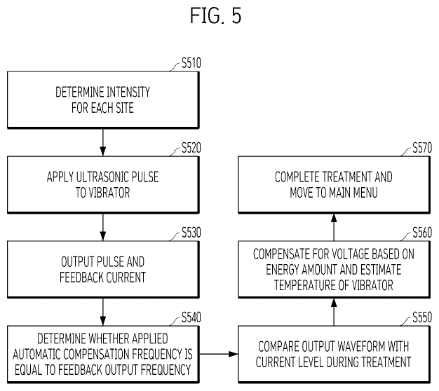

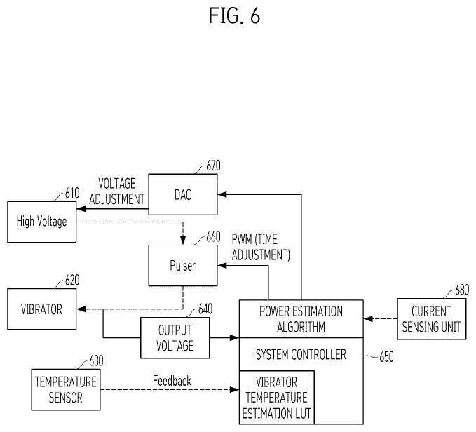

FIG. 5 is a flowchart illustrating a method for controlling a device according to an embodiment of the present disclosure.

First, as shown in FIG. 5, the device according to an embodiment of the present disclosure determines an intensity for each site to which a patch is attached for a treatment or the like (S510).

An ultrasonic pulse is applied to a vibrator included in the device (S520).

Unlike in prior art, a feedback result for an output and a current of the applied ultrasonic pulse is monitored (S530).

For example, a controller (the system controller described above) determines whether an applied automatic compensation frequency is equal to a feedback output frequency (S540).

When the frequencies are not equal to each other, the controller compares an output waveform and a current level applied to the ultrasonic vibrator with each other (S550).

Further, the device compensates for a voltage applied to the ultrasonic vibrator in consideration of a target energy amount, and estimates a temperature of the ultrasonic vibrator with reference to an LUT (S560).

When a treatment time elapses, the device automatically moves to a main menu (S570).

In other words, a value measured by a temperature sensor after a user attaches the patch to skin and immediately before the treatment starts is designed to be regarded as an initial temperature.

After storing the initial temperature, the device is controlled by estimating a skin surface temperature based on the treatment time (e.g., 20 minutes for one treatment), taking into account a temperature increase over time by a frequency and a voltage in the LUT.

When a feedback voltage is reduced and the skin measurement temperature is predicted to exceed 43 degrees during the treatment time, the device is designed to resume the treatment again after a cool down time.

The vibrator temperature estimation LUT is a table representing a temperature table based on a vibrator optimal operating frequency and a measured voltage value of the ultrasonic output. That is, the frequency and the voltage are input values, and a table corresponding thereto is able to be an example of the LUT.

Further, FIG. 6 illustrates an embodiment of a device that uses frequency shifting for a resonant frequency.

As illustrated in FIG. 6, the device according to an embodiment of the present disclosure includes, for example, a high voltage generator 610, a vibrator 620, a temperature sensor 630, an output voltage 640, a system controller 650, a pulser 660, a DAC 670, a current sensing unit 680, and the like.

When it is difficult to adjust a treatment time with the device according to an embodiment of the present disclosure, a voltage and a current of an output waveform are measured using a frequency shifting scheme, and then an output voltage applied to the ultrasonic vibrator is corrected using an output power estimation algorithm using three values (frequency, current, and output voltage).

The device according to an embodiment of the present disclosure determines a final frequency and the output voltage by comparing the power estimation algorithm with a skin surface temperature estimated by the temperature sensor.

Furthermore, when it is difficult to control the temperature with reference to an LUT or when an irradiation time is extended for 5 minutes or more, the device according to an embodiment of the present disclosure stores an output voltage and a current value obtained by performing the frequency shifting in units of 3 to 5 Khz within a range of +−10% of a resonant frequency of the ultrasonic vibrator, and performs the treatment by reflecting the output power estimation algorithm of a treatment available range on the current vibrator.

In case of the power estimation algorithm, a phase is calculated for a waveform and a current within the range of +−10% of the resonant frequency, an output voltage and a current input based thereon are substituted into an algorithm formula to estimate a frequency and a target output having the smallest temperature change, and the estimated frequency and target output are reflected in the device.

In other words, the present disclosure is applicable to any type of device including at least one ultrasonic vibrator.

The device according to an embodiment of the present disclosure includes at least one ultrasonic vibrator (e.g., 231 shown in FIG. 2), a pulser (e.g., 220 shown in FIG. 2) that creates an ultrasonic waveform, and a controller (e.g., 270 shown in FIG. 2).

The controller 270 recognizes whether the at least one ultrasonic vibrator is abnormal, and determines whether target energy compensation of the at least one ultrasonic vibrator 231 is required when there is no abnormality as a result of the recognition.

Further, when the compensation is required as a result of the determination, the controller 270 increases a voltage input to the pulser 220, increases a PWM duty ratio, or increases an irradiation time of the ultrasonic vibrator 231.

Furthermore, when a difference between the voltage input to the pulser 220 and a voltage applied to the ultrasonic vibrator 231 is equal to or greater than a first reference value (e.g., 10), the controller 270 estimates that the abnormality has occurred in the ultrasonic vibrator 231.

In addition, the controller 270 forcibly stops operation of the ultrasonic vibrator 231 when a surface temperature of the ultrasonic vibrator 231 is estimated to rise to a second reference value (e.g., 43 degrees Celsius) or higher based on an LUT stored in a memory. The LUT or the like described above may be used.

However, when the surface temperature of the ultrasonic vibrator 231 is not lowered to a target temperature within a preset target time (e.g., within 5 minutes), the controller 270 adjusts the pulser 220 with a voltage or a current obtained via the frequency shifting in units of about 3 to 5 Khz within a range in which-10% of the resonant frequency of the ultrasonic vibrator 231 has a minimum value and +10% thereof has a maximum value. When it is out of the above range, a speed of lowering the surface temperature is lowered.

As another embodiment, the present disclosure may be applied to a cosmetic care device.

The cosmetic care device includes the head 230 and the main body.

The head 230 includes at least one skin temperature sensor 232 and at least one hifu and Flat ultrasonic vibrator 231.

In one example, the main body includes the pulser 220 that creates the ultrasonic waveform and the controller 270 that controls the ultrasonic vibrator 231 included in the head 230.

When it is predicted that a temperature of a specific ultrasonic vibrator among a plurality of ultrasonic vibrators will be equal to or higher than a predetermined temperature (e.g., 43 degrees Celsius), the controller 270 may lower a voltage input to the pulser 220 and increase an irradiation time of the specific ultrasonic vibrator such that the specific ultrasonic vibrator is operated at a temperature lower than the predetermined temperature.

When an irradiation stop time of the specific ultrasonic vibrator is 5 minutes or longer, the controller 270 additionally adjusts the pulser 220 with a voltage or a current obtained via the frequency shifting in units of 3 to 5 Khz within a range in which-10% of a resonant frequency of the specific ultrasonic vibrator has a minimum value and +10% thereof has a maximum value. Therefore, there is an advantage in that the high-temperature ultrasonic vibrator may be cool down more quickly.

The present disclosure described above may be implemented as computer-readable codes in a medium in which a program is recorded. A computer-readable medium includes all kinds of recording devices in which data readable by a computer system is stored. Examples of the computer-readable medium include an application, a hard disk drive (HDD), a solid state disk (SSD), a silicon disk drive (SDD), a ROM, a RAM, a CD-ROM, a magnetic tape, a floppy disk, an optical data storage device, and the like, and also include a carrier wave (e.g., transmission via the Internet).

Although the device and the method for processing data according to an embodiment of the present disclosure have been described as specific embodiments, this is merely an example, and the present disclosure is not limited thereto and should be interpreted as having the best range according to the basic idea disclosed herein. Those skilled in the art may combine or substitute the disclosed embodiments to implement an embodiment not specified, but this also does not deviate from the scope of the present disclosure. In addition, those skilled in the art may easily change or modify the disclosed embodiments based on the present document, and it is clear that such changes or modifications also fall within the scope of the present disclosure.

### Mode

Various forms (embodiments) for implementing the present disclosure have been described in detail in the previous table of contents, so that even when redundant descriptions are omitted, those skilled in the art may implement various modifications.

### Industrial Availability

The present disclosure is applicable to any type of device (a cosmetic care device, a body care device, a treatment device, and the like) including an ultrasonic vibrator, so that industrial applicability thereof is recognized.

What is claimed is:

1. A method for controlling a device including at least one ultrasonic vibrator, the method comprising:
recognizing whether the at least one ultrasonic vibrator is abnormal;
determining whether target energy compensation of the at least one ultrasonic vibrator is required when no abnormality is found as a result of the recognition; and
increasing a voltage input to a pulser, increasing a pulse width modulation (PWM) duty ratio, or increasing an irradiation time when the compensation is required as a result of the determination.

2. The method of claim 1, wherein the recognizing of whether the at least one ultrasonic vibrator is abnormal includes:
estimating that the abnormality has occurred in the at least one ultrasonic vibrator when a difference between the voltage input to the pulser and a voltage applied to the at least one ultrasonic vibrator is equal to or greater than a first reference value.

3. The method of claim 2, further comprising:
forcibly stopping operation of the at least one ultrasonic vibrator when it is estimated that a surface temperature of the at least one ultrasonic vibrator will rise to a second reference value or higher based on a look-up table (LUT) stored in a microcontroller unit (MCU) and a flash memory.

4. The method of claim 3, wherein the first reference value is 10V and the second reference value is 43 degrees Celsius.

5. The method of claim 1, further comprising:
adjusting the pulser with a voltage or a current obtained via frequency shifting in units of 3 to 5 Khz within a range of +/−10% of a resonant frequency of the at least one ultrasonic vibrator when a surface temperature of the at least one ultrasonic vibrator is not lowered to a target temperature within a predetermined target time.

6. A device comprising:
at least one ultrasonic vibrator;
a pulser configured to create an ultrasonic waveform; and
a controller configured to:
recognize whether the at least one ultrasonic vibrator is abnormal;
determine whether target energy compensation of the at least one ultrasonic vibrator is required when no abnormality is found as a result of the recognition; and
increase a voltage input to the pulser, increase a PWM duty ratio, or increase an irradiation time when the compensation is required as a result of the determination.

7. The device of claim 6, wherein the controller is configured to estimate that the abnormality has occurred in the at least one ultrasonic vibrator when a difference between the voltage input to the pulser and a voltage applied to the at least one ultrasonic vibrator is equal to or greater than a first reference value.

8. The device of claim 7, wherein the controller is configured to forcibly stop operation of the at least one ultrasonic vibrator when it is estimated that a surface temperature of the at least one ultrasonic vibrator will rise to a second reference value or higher based on a look-up table (LUT) stored in a memory.

9. The device of claim 8, wherein the first reference value is 10V and the second reference value is 43 degrees Celsius.

10. The device of claim 6, wherein the controller is configured to adjust the pulser with a voltage or a current obtained via frequency shifting in units of 3 to 5 Khz within a range of +/−10% of a resonant frequency of the at least one ultrasonic vibrator when a surface temperature of the at least one ultrasonic vibrator is not lowered to a target temperature within a predetermined target time.

11. A cosmetic care device comprising:
a head including at least one skin temperature sensor and at least one ultrasonic vibrator; and
a main body including a pulser configured to create an ultrasonic waveform, and a controller configured to control the at least one ultrasonic vibrator included in the head,
wherein the controller is configured to, when a specific ultrasonic vibrator among the at least one ultrasonic vibrator is predicted to reach a temperature equal to or higher than a predetermined temperature, lower a voltage input to the pulser and increase an irradiation time of the specific ultrasonic vibrator such that the specific ultrasonic vibrator is operated at a temperature lower than the predetermined temperature.

12. The cosmetic care device of claim 11, wherein the predetermined temperature corresponds to 43 degrees Celsius.

13. The cosmetic care device of claim 12, wherein the controller is configured to adjust the pulser with a voltage or a current obtained via frequency shifting in units of 3 to 5 Khz within a range having a minimum value of −10% of a resonant frequency of the specific ultrasonic vibrator and a maximum value of +10% of the resonant frequency when the irradiation time of the specific ultrasonic vibrator is equal to or greater than 5 minutes.
